(19) United States
(12) Patent Application Publication
(10) Pub. No.: US 2020/0248218 A1
(43) Pub. Date: Aug. 6, 2020

(54) A RECOMBINANT MICROORGANISM PRODUCING L-THREONINE AND A METHOD FOR PRODUCING L-THREONINE USING THE SAME

(71) Applicant: CJ CHEILJEDANG CORPORATION, Seoul (KR)

(72) Inventors: Chang Il Seo, Incheon (KR); Hye Min Park, Suwon-si (KR); Kwang Ho Lee, Daejeon (KR); Sang Min Park, Suwon-si (KR); Kyung Rim Kim, Seoul (KR); Ki Yong Cheong, Suwon-si (KR)

(73) Assignee: CJ CHEILJEDANG CORPORATION, Seoul (KR)

(21) Appl. No.: 15/769,920

(22) PCT Filed: Oct. 21, 2016
(86) PCT No.: PCT/KR2016/011907

§ 371 Date: Apr. 20, 2018

(30) Foreign Application Priority Data
Oct. 23, 2015 (KR) 10-2015-0148004

(51) Int. Cl. C12P 13/08; C12N 9/88; C12N 9/10

(52) U.S. Cl. CPC C12P 13/08; C12N 9/88; C12N 9/1029; C12Y 203/01182; C12Y 403/01019

(57) ABSTRACT

The present disclosure relates to a recombinant microorganism producing threonine, and a method for producing L-threonine using the same.

### TECHNICAL FIELD

The present disclosure relates to a recombinant microorganism producing threonine, and a method for producing L-threonine using the same.

### BACKGROUND ART

Threonine, an essential amino acid, is widely used in feeds, food additives, and animal growth promoters, and is also effectively used in rehydration solutions and synthetic materials for medical and pharmaceutical uses.

With regard to a method for producing threonine using a microorganism, methods for enhancing threonine biosynthesis genes (e.g., ppc, aspC, and thrABC) and for blocking a threonine degradation pathway are known for increasing the yield (Kwang-Ho Lee, et al., Molecular System Biology 2007). The genes involved in the threonine degradation pathway include tdh, tdcB, glyA, ilvA, etc., and among these, threonine deaminase (ilvA) is known to be the most important threonine degradation gene. The deletion of ilvA among degradation genes greatly improves the yield of threonine, but is problematic in that an expensive isoleucine auxotroph appears; therefore, it is generally known to apply attenuation of the ilvA activity and high-susceptibility mutation for isoleucine (Akhverdian Valery Z, et al.).

Meanwhile, a strain of the genus Corynebacterium or a strain of the genus Escherichia produces 2-ketobutyrate and biosynthesizes isoleucine through ilvA, which is the gene involved in the threonine degradation pathway. However, it is known that a microorganism such as Methanococcus jannaschii can synthesize 2-ketobutyrate and isoleucine using acetyl-CoA and pyruvate as substrates through citramalate synthase (cimA) (Atsumi, Liao J C, Appl Environ Microbiol., 2008).

### DISCLOSURE

### Technical Problem

The present inventors have attempted to develop a method for effectively producing threonine. As a result, they have confirmed that the yield of threonine production was improved by a method of reducing the activity of the threonine deaminase (IlvA) enzyme in the threonine-producing strain, and by a method of introducing the gene of citramalate synthase (cimA), and have also confirmed that the existing problem (Xie X, et al., J Ind Microbiol Biotechnol. 2014), wherein the culture is delayed due to accumulation of acetate, could be overcome, thereby completing the present disclosure.

### Technical Solution

An object of the present disclosure is to provide a recombinant microorganism that produces threonine with high efficiency.

Another object of the present disclosure is to provide a method for producing threonine using the microorganism above.

### Advantageous Effects

The recombinant microorganism of the present disclosure, which produces threonine, maintains or improves its growth ability and has excellent threonine productivity because the amount of acetate accumulation in the microorganism is reduced by reducing or inactivating the activity of threonine deaminase (ilvA) and by introducing the activity of citramalate synthase (cimA). Accordingly, the microorganism can be widely used for the efficient and economical production of threonine even on an industrial scale.

### BRIEF DESCRIPTION OF DRAWING

FIG. 1 is a schematic diagram showing a gene map of a pCJ-MuAB plasmid, which is a helper plasmid used in a mini-Mu gene insertion method.

FIG. 2 is a schematic diagram showing a gene map of a pMu-R6K plasmid, which is an integrative plasmid used in a mini-Mu gene insertion method that is used for introduction of a threonine biosynthesis gene.

### BEST MODE

In order to achieve the objects above, an aspect of the present disclosure provides a recombinant microorganism producing threonine, wherein an activity of threonine deaminase (IlvA) is reduced or inactivated and an activity of citramalate synthase is retained.

As used herein, the term “threonine” refers to an amino acid with the formula HO₂CCH(NH₂)CH(OH)CH₃, which is a hydroxy-a-amino acid, one of the essential amino acids that are not produced in the body. In the present disclosure, threonine may be an L- or D-type optical isomer, but is not limited thereto.

As used herein, the term “threonine productivity” refers to an ability of a microorganism to produce and accumulate threonine in the microorganism or in the medium in which the microorganism is cultured. Such threonine productivity may be a property retained by a wild-type strain or a property imparted or enhanced by modification of the strain.

As used herein, the term “threonine deaminase (EC4.3.1.19)” may be referred to as threonine ammonia-lyase or threonine dehydratase, and is an enzyme catalyzing a reaction of converting threonine to a-ketobutyrate and ammonia. The a-ketobutyrate produced therefrom is used as a precursor of isoleucine. Therefore, when the threonine deaminase is deficient, the biosynthesis of isoleucine becomes abnormal, and thus the growth of microorganisms may be retarded.

As used herein, the terms “threonine deaminase enzyme” and “gene encoding the same” may include any proteins having the activity of threonine deaminase and any genes encoding the same, but these are not limited thereto.

Specifically, the threonine deaminase may be easily obtained from a known database such as the National Center for Biotechnology Information (NCBI) or the DNA Data Bank of Japan (DDBJ), and for example, it may be represented by the amino acid sequence of SEQ ID NO: 7. However, because the amino acid sequence of the protein showing the activity may differ depending on species or strain of the microorganism, the protein is not limited thereto.

Additionally, if the protein is a protein substantially having the activity of threonine deaminase, and if the protein is composed of an amino acid sequence having 70% or more homology to an amino acid sequence of SEQ ID NO: 7, specifically 80% or more homology thereto, more specifically 90% or more homology thereto, much more specifically 95% or more homology thereto, much more specifically 98% or more homology thereto, and most specifically 99% or more homology thereto, the protein can be included in the scope of the present disclosure without limitation.

Further, it is apparent that an amino acid sequence having homology to the sequence above, part of which is deleted, modified, substituted, or added, is also within the scope of the present disclosure, as long as the resulting amino acid sequence has a biological activity substantially equivalent or corresponding to the protein of SEQ ID NO: 7.

Additionally, the gene encoding the threonine deaminase may have a nucleotide sequence encoding the amino acid sequence of SEQ ID NO: 7. In the polynucleotide, various modifications may be made in the coding region without changing an amino acid sequence of the protein, due to codon degeneracy or in consideration of the codons preferred in an organism in which the protein is to be expressed. The polynucleotide sequence may have, for example, a polynucleotide sequence of SEQ ID NO: 30 and may have a nucleotide sequence having 80% homology thereto, specifically 90% homology thereto, and more specifically 99% homology thereto, but is not limited thereto.

As used herein, the term “homology” refers to a degree of matching with a given amino acid sequence or nucleotide sequence, and the homology may be expressed as a percentage. In the present disclosure, a homology sequence having an activity which is identical or similar to the given amino acid sequence or nucleotide sequence is expressed as “% homology”. For example, the % homology may be confirmed using standard software, i.e., BLAST 2.0, for calculating parameters such as score, identity, and similarity, or by comparing sequences via southern hybridization experiments, and the appropriate hybridization condition to be defined may be determined by a method known to those skilled in the art (e.g., Sambrook et al., Molecular Cloning: A Laboratory Manual 1989).

As used herein, the term “reduction of enzyme activity” means that the activity of the relevant enzyme is reduced relative to an endogenous activity of the wild-type or state before modification.

Additionally, as used herein, the term “inactivation” means that the gene encoding the relevant enzyme is expressed at a low level or is not expressed, compared to that of a wild-type strain, or the activity is eliminated or reduced, even though the gene is expressed. Reduction of inactivation of the enzyme activity may be accomplished by any methods known in the art.

Specifically, the inactivation may be carried out by deletion of a part or the entirety of a polynucleotide encoding the enzyme; modification of an expression regulatory sequence for reducing expression of the polynucleotide; modification of the polynucleotide sequence on the chromosome to weaken an activity of the protein; or by a method selected from a combination thereof, but is not particularly limited thereto.

A partial or entire deletion of a polynucleotide encoding the protein may be carried out by substituting the polynucleotide encoding an endogenous target protein in the chromosome with a marker gene or a polynucleotide in which a partial nucleotide sequence was deleted, with a vector for chromosomal gene insertion.

Additionally, in order to reduce or eliminate the polynucleotide expression, an expression regulatory sequence may be modified by inducing variations in the expression regulatory sequence through deletion, insertion, conservative or non-conservative substitution of nucleotide sequence, or a combination thereof to further weaken the activity of the expression regulatory sequence, or by replacing the expression regulatory sequence with the nucleotide sequence having a weaker activity, but is not particularly limited thereto. The expression regulatory sequence may include a sequence encoding promoter, operator sequence, ribosomal binding site, and a sequence controlling the termination of transcription and translation, but is not limited thereto.

Further, modification of a polynucleotide sequence on the chromosome may be performed by inducing variations on the sequence through deletion, insertion, non-conservative or conservative substitution of the polynucleotide sequence, or a combination thereof in order to further attenuate the protein activity, or by replacing the sequence with a polynucleotide sequence which is modified to have a weaker activity, but is not limited thereto.

As used herein, the term “recombinant vector” refers to a DNA construct containing a nucleotide sequence of a polynucleotide encoding a target protein operably linked to a suitable regulatory sequence such that the target protein is expressed in a suitable host cell. The regulatory sequence may include a promoter capable of initiating transcription, any operator sequence for regulating such transcription, a sequence encoding a suitable mRNA ribosome-binding site, and a sequence for regulating the termination of transcription and translation. After being transformed into a suitable host cell, the recombinant vector may be replicated or perform its function irrespective of the host genome or may be integrated into the genome itself.

The recombinant vector used in the present disclosure is not particularly limited as long as it is replicable in a host cell, and may be produced using any vector known in the art. Examples of a commonly used vector include a natural or recombinant plasmid, a cosmid, a virus, and a bacteriophage. For example, pWE15, M13, MBL3, MBL4, IXII, ASHII, APII, t10, t11, Charon4A, and Charon21A may be used as a phage vector or a cosmid vector; and pBR-based, pUC-based, pBluescriptII-based, pGEM-based, pTZ-based, pCL-based, and pET-based may be used as a plasmid vector. The vectors that can be used in the present disclosure are not particularly limited, and expression vectors known in the art may be used. Specifically, pECCG117, pDZ, pACYC177, pACYC184, pCL, pUC19, pBR322, pMW118, and pCC1BAC vectors may be used.

For example, a polynucleotide encoding a target protein may be introduced into a chromosome through a vector for chromosomal insertion into a host cell. The introduction of the polynucleotide into a chromosome may be carried out by any method known in the art, for example, by homologous recombination, but is not limited thereto.

As used herein, the term “transformation” means that a vector containing a polynucleotide encoding a target protein is introduced into a host cell, such that the protein encoded by the polynucleotide is capable of being expressed in the host cell. The transformed polynucleotide may be any one regardless of the position as long as the polypeptide is capable of being expressed in the host cell, regardless of whether the polynucleotide is inserted and positioned into the chromosome of the host cell or positioned on an outer portion of the chromosome.

Additionally, the term “operably linked” refers to a functional connection between a promoter sequence, which initiates and mediates the transcription of the polynucleotide encoding the target protein of the present disclosure, and the gene sequence.

As used herein, the term “citramalate synthase (EC2.3.1.182)” refers to an enzyme that catalyzes the conversion of acetyl-CoA and pyruvate to citramalate and coenzyme A. This enzyme has been discovered in Archaea, such as Methanococcus jannaschii (Howell et al., J Bacteriol. 181: 331-333, 1999), Leptospira interogans (Xu et al., J Bacteriol. 186: 5400-5409, 2004), Geobacter sulfurreducens, etc., and involves in the threonine-independent pathway of isoleucine biosynthesis (Risso et al., J Bacteriol. 190: 2266-2274, 2008). The enzyme is highly specific for pyruvate as a substrate and is typically inhibited by isoleucine.

As used herein, the terms “citramalate synthase enzyme” and “gene encoding the same” include, without limitation, any protein having the activity of citramalate synthase and any gene encoding the same. The sequence of the enzyme can be easily obtained from a known database such as the National Center for Biotechnology Information (NCBI) or the DNA Data Bank of Japan (DDBJ).

Specifically, the citramalate synthase enzyme may be citramalate synthase derived from Methanococcus jannaschii and may be represented by an amino acid sequence selected from the group consisting of SEQ ID NOS: 1, 2, and 3.

Additionally, cimA, which is the gene encoding the citramalate synthase, may have a nucleotide sequence encoding an amino acid sequence selected from the group consisting of SEQ ID NOS: 1, 2, and 3. Specifically, the polynucleotide sequence may have, for example, a nucleotide sequence selected from the group consisting of SEQ ID NOS: 4, 5, and 6, and may have a nucleotide sequence having 80% or more homology thereto, specifically 90% or more homology thereto, and more specifically 99% or more homology thereto, but is not limited thereto. A difference may exist in the amino acid sequence of the protein exhibiting the activity depending on the species or strain of the microorganism, or because of the degeneracy of the genetic code, a large number of functionally identical nucleic acids may encode any given protein; therefore, the polynucleotide sequence is not limited thereto.

Additionally, if the protein is a protein substantially having the activity of citramalate synthase, and if the protein is an amino acid sequence having 70% or more homology to an amino acid sequence selected from the group consisting of SEQ ID NOS: 1, 2, and 3, specifically 80% or more homology thereto, more specifically 90% or more homology thereto, much more specifically 95% or more homology thereto, much more specifically 98% or more homology thereto, and most specifically 99% or more homology thereto, the protein can be included within the scope of the present disclosure without limitation.

Further, it is apparent that an amino acid sequence having homology to the sequence above, the part of which is deleted, modified, substituted, or added, is also within the scope of the present disclosure, as long as the resulting amino acid sequence has a biological activity substantially equivalent or corresponding to the protein of the amino acid selected from the group consisting of SEQ ID NOS: 1, 2, and 3.

As used herein, the term “recombinant microorganism” may include, without limitation, a variant microorganism in which a gene is manipulated. If genetic manipulation is possible, the recombinant microorganism may be a microorganism of the genus Escherichia, a microorganism of the genus Corynebacterium, a microorganism of the genus Brevibacterium, a microorganism of the genus Serratia, or a microorganism of the genus Providencia, but is not limited thereto. For example, the recombinant microorganism of the present disclosure may be a microorganism of the genus Corynebacterium or a microorganism of the genus Escherichia, and specifically the microorganism of the genus Corynebacterium may be Corynebacterium glutamicum, and the microorganism of the genus Escherichia may be Escherichia coli.

In the present disclosure, the recombinant microorganism may have a parent strain having natural threonine productivity, or may have a parent strain having a variant strain in which a variation is introduced in order to enhance threonine productivity. The variation that enhances threonine productivity may be, for example, performed by enhancing genes associated with threonine biosynthesis or by attenuating genes associated with the threonine degradation pathway. The genes associated with threonine biosynthesis may be one or more genes selected from the group consisting of pntAB encoding pyridine nucleotide transhydrogenase, asd encoding aspartate-β-semialdehyde dehydrogenase, aspC encoding aspartate aminotransferase, gdhA encoding glutamate dehydrogenase, ppc encoding phosphoenol pyruvate carboxylase, aspA encoding aspartase, threonine operon (thrO), etc.; and the genes associated with the threonine degradation pathway may be one or more genes selected from the group consisting of tdh encoding threonine dehydrogenase, tdcB encoding catabolic threonine hydratase, etc.

Another aspect of the present disclosure provides a method for producing threonine, comprising: culturing the recombinant microorganism of the present disclosure in a medium; and recovering threonine from the microorganism or the cultured medium.

Specifically, the present disclosure provides a method for producing threonine, comprising: culturing a recombinant microorganism producing threonine, in which the activity of threonine deaminase is reduced or inactivated and the activity of citramalate synthase is introduced, in a medium; and recovering threonine from the microorganism or the cultured medium.

In the present disclosure, the recombinant microorganism producing threonine deaminase, citramalate synthase, and threonine is as described above.

As used herein, the term “culture” refers to culturing of a microorganism under artificially controlled environmental conditions. In the present disclosure, the step of culturing a microorganism, e.g., a microorganism of the genus Corynebacterium or a microorganism of the genus Escherichia, may be carried out using any culture conditions and culture methods known in the art. In the method above, culturing of the microorganism of the present disclosure, e.g., a microorganism of the genus Corynebacterium or a microorganism of the genus Escherichia, may be carried out by, although is not particularly limited to, batch culture, continuous culture, and fed-batch culture known in the art.

The medium used for the culture must satisfy the requirements for a particular strain in an appropriate manner, and can be suitably used by those skilled in the art. The culture medium for a strain of the genus Corynebacterium or a strain of the genus Escherichia is known in the art (e.g., Manual of Methods for General Bacteriology. American Society for Bacteriology. Washington D.C., USA, 1981).

In particular, for the culture conditions, an appropriate pH (e.g., a pH of 5 to 9, preferably a pH of 6 to 8, and most preferably a pH of 6.8) can be adjusted using a basic compound (e.g., sodium hydroxide, potassium hydroxide, or ammonia) or an acidic compound (e.g., phosphoric acid or sulfuric acid), but is not particularly limited thereto. In addition, bubble formation can be inhibited by using an anti-foaming agent, such as a fatty acid polyglycol ester.

Oxygen or an oxygen-containing gas mixture can be introduced into the culture to maintain aerobic conditions, and the temperature of the culture is usually 20° C. to 45° C., preferably 25° C. to 40° C. The cultivation continues until the maximum amount of threonine produced is obtained, and usually takes 10 hours to 160 hours to achieve the same. Threonine may be released into the culture medium or may be contained in the cells.

Further, the culture medium used may comprise a sugar and carbohydrate (e.g., glucose, sucrose, lactose, fructose, maltose, molasses, starch, and cellulose), oil and fat (e.g., soybean oil, sunflower seed oil, peanut oil, and coconut oil), fatty acid (e.g., palmitic acid, stearic acid, and linoleic acid), alcohol (e.g., glycerol and ethanol), and organic acid (e.g., acetic acid) individually or in combination as a carbon source, but the medium is not limited thereto.

In addition, the culture medium may comprise a nitrogen-containing organic compound (e.g., peptone, yeast extract, meat juice, malt extract, corn solution, soybean meal powder, and urea), or an inorganic compound (e.g., ammonium sulfate, ammonium chloride, ammonium phosphate, ammonium carbonate, and ammonium nitrate) individually or in combination as a nitrogen source, but the medium is not limited thereto.

In addition, the culture medium may comprise potassium dihydrogen phosphate, dipotassium hydrogen phosphate, or a sodium-containing salt corresponding thereto individually or in combination as a phosphorous source, but the medium is not limited thereto.

Additionally, the culture medium may comprise other essential growth-stimulating substances including metal salts (e.g., magnesium sulfate or iron sulfate), amino acids, and vitamins, but the medium is not limited thereto.

Additionally, the culture medium may contain suitable precursors. These substances may be added to the medium during culturing in a batch or continuous manner, but are not limited thereto.

Further, recovery of threonine from the microorganism or cultured medium may be carried out by a suitable method known in the art, which is selected depending on the culture method, e.g., a batch, continuous, or fed-batch culture method. Specifically, the threonine-recovering method known in the art may include centrifugation, filtration, extraction, spraying, drying, evaporation, precipitation, crystallization, electrophoresis, fractional dissolution (e.g., ammonium sulfate precipitation), chromatography (e.g., HPLC, ion exchange, affinity, hydrophobicity, and size exclusion), etc., but is not limited thereto.

Still another aspect of the present disclosure provides the use of a recombinant microorganism, in which an activity of threonine deaminase (IlvA) is reduced or inactivated and an activity of citramalate synthase is retained, for production of threonine.

In such use, the recombinant microorganism producing threonine, in which an activity of threonine deaminase (IlvA) is reduced or inactivated and an activity of citramalate synthase is retained, is as described above.

As described above, the recombinant microorganism of the present disclosure, which produces threonine, maintains or improves its growth ability and has excellent threonine productivity because the amount of acetate accumulation in the microorganism is reduced by reducing or inactivating the activity of threonine deaminase (ilvA) and by introducing the activity of citramalate synthase (cimA).

### DETAILED DESCRIPTION OF THE EMBODIMENT

Hereinbelow, the present disclosure will be described in detail with accompanying exemplary embodiments. However, the exemplary embodiments disclosed herein are only for illustrative purposes and should not be construed as limiting the scope of the present disclosure.

### EXAMPLE 1

### Preparation of Wild-Type E. coli-Based Threonine-Producing Strain

### 1-1. Preparation of Plasmid for Gene Insertion and Helper Plasmid

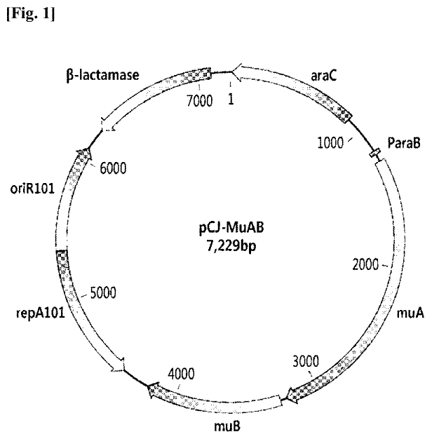

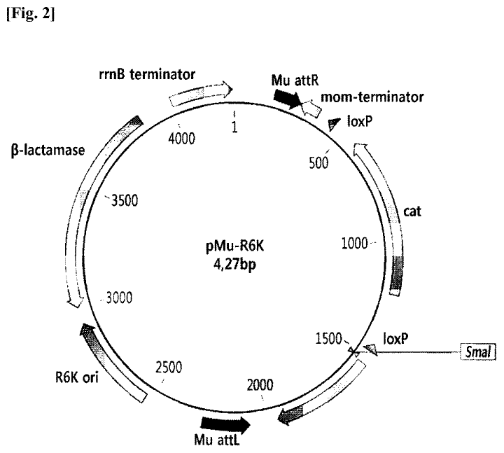

A mini-Mu transposon-based genomic insertion method (Akhverdyan V Z, Gak E R et al., Appl Microbiol Biotechnol. 2011) was used to produce threonine from wild-type E. coli W3110 (Accession Number: ATCC9637). pCJ-MuAB (helper plasmid; FIG. 1) and pMu-R6K (integrative plasmid; FIG. 2) were prepared in order to utilize the mini-Mu insertion method. In pCJ1-MuAB, the helper plasmid, transposition factors MuA and MuB were expressed using an arabinose-inducible ParaB promoter. In addition, the helper plasmid was designed to easily eliminate plasmids because it contained temperature-sensitive replicons. pMu-R6K, the integrative plasmid, had the gene, cat, including Mu-attL/R, which is a mini-Mu unit, and loxP66 and loxP71 on both ends (Oleg Tolmachov, et al., Biotechnol. 2006). In addition, since the integrative plasmid had R6K replicons, the plasmid had a property of a suicidal vector, in which proliferation does not occur in normal E. coli without the gene pir (Xiao-Xing Wei, Zhen-Yu Shi. et al., Appl Microbiol Biotechnol. 2010).

### 1-2. Cloning of Integrative Plasmid for Enhancing Threonine Biosynthesis Gene

Three integrative plasmids were prepared to enhance expression of the threonine biosynthesis gene using the plasmids prepared in Example 1-1 above.

### 1-2-1: pMu-R6K_pntAB asd aspC-thrO

pMu-R6K, the integrative plasmid prepared in Example 1-1 above, was treated with SmaI, a restriction enzyme, followed by DNA purification. Thereafter, a truncated linear cloning vector was prepared. The gene, pntAB, was amplified by PCR using W3110 genomic DNA as a template and SEQ ID NOS: 8 and 9 as primers. Specifically, denaturation was performed at 94° C. for 30 seconds, annealing at 55° C. for 30 seconds, and extension at 72° C. for 3 minutes using SolGent™ Pfu-X DNA Polymerase (SolGent, DNA); and this was repeated 30 times.

Additionally, the gene, asd, was amplified as described above; that is, PCR was performed using W3110 genomic DNA as a template and SEQ ID NOS: 10 and 11 as primers. In addition, the gene, aspC, was amplified by PCR using W3110 genomic DNA as a template and SEQ ID NOS: 12 and 13 as primers.

The threonine operon (thrO) was amplified from genomic DNA of KCCM10541 (Korean Patent No. 10-0576342) by PCR using SEQ ID NOS: 14 and 15 as primers. Each of the four DNAs and vector DNAs prepared in the above underwent a Gibson assembly method to prepare the plasmid, pMu-R6K_pntAB asd aspC-thrO (Daniel G Gibson, et al., Nature Methods 2009).

In the Gibson assembly method, 5× ISO buffer was prepared using the composition shown in Table 1 below, and then the Gibson assembly master mix (15 μL), in which the 5× ISO buffer was mixed with the three enzymes (e.g., T5 exonuclease (Epicentre), Phusion polymerase (New England Biolabs), and Taq ligase (New England Biolabs)) using the composition shown in Table 2 below, was reacted with the DNAs (5 μL) to be cloned at 50° C. for 1 hour. Thereafter, electroporation (2500 V) was applied to competent cells of TransforMax™ EC 100D™ pir-116 Electrocompetent E. coli (Epicentre, USA). The strains were recovered therefrom, plated on LB plate medium containing ampicillin (100 μg/L), and then cultured overnight at 37° C. Thereafter, the strains showing resistance to ampicillin were selected. A plasmid was obtained from the selected strains, and whether it had a desired nucleotide sequence was determined by sequencing analysis.

TABLE 1
Composition of 5× ISO buffer

| Component | Content | |
|---|---|---|
| 1 M Tris-HCl pH 7.5 | 3 | mL |
| 2 M MgCl₂ | 150 | μL |
| 100 nM dNTP mix | 240 | μL |
| 1 M DTT | 300 | μL |
| PEG-8000 | 1.5 | g |
| 100 mM NAD | 300 | μL |

| | |
|---|---|
| Total | dH₂O up to 6 mL |

dH₂O distilled water.

TABLE 2
Composition of Gibson assembly master mix

| Component | Content | |
|---|---|---|
| 5× ISO buffer | 320 | μL |
| 10 U/μL T5 exonuclease | 0.64 | μL |
| 2 U/μL Phusion polymerase | 20 | μL |
| 40 U/μL Tag ligase | 160 | μL |

| | |
|---|---|
| Total | dH₂O up to 1.2 mL |

### 1-2-2: pMu-R6K_gdhA aspC thrO

pMu-R6K, the integrative plasmid prepared in Example 1-1, was treated with SmaI, a restriction enzyme, in the same manner as in Example 1-2-1, followed by DNA purification. Thereafter, a truncated linear cloning vector was prepared. From W3110 genomic DNA, the gene, gdhA, was amplified by PCR using SEQ ID NOS: 18 and 19 as primers, and the gene, aspC, was amplified by PCR using SEQ ID NOS: 20 and 21 as primers. The threonine operon (thrO) was amplified from KCCM10541 genomic DNA by PCR using SEQ ID NOS: 14 and 15 as primers. Each of the three DNAs and vectors DNAs prepared in the above underwent the Gibson assembly method to prepare the plasmid, pMu-R6K_gdhA aspC thrO.

### 1-2-3: pMu-ppc aspA thrO

pMu-R6K, the integrative plasmid prepared in Example 1-1 above, was treated with SmaI, a restriction enzyme, in the same manner as in Example 1-2-1, followed by DNA purification. Thereafter, a truncated linear cloning vector was prepared. From W3110 genomic DNA, the gene, ppc, was amplified by PCR using SEQ ID NOS: 22 and 23 as primers, and the gene, aspA, was amplified by PCR using SEQ ID NOS: 24 and 25 as primers. The threonine operon (thrO) was amplified from KCCM10541 genomic DNA by PCR using SEQ ID NOS: 26 and 27 as primers. Each of the three DNAs and vector DNAs prepared in the above underwent the Gibson assembly method to prepare the plasmid, pMu-R6K_ppc aspA thrO.

### 1-3. Preparation of Threonine Biosynthesis-Enhanced Wild-Type E. coli

Wild-type E. coli W3110, in which pCJ-MuAB prepared in Example 1-1 above was transformed, was cultured at 30° C. until OD₆₀₀ reached 0.6 using LB medium supplemented with ampicillin (100 μg/L) and 5 mM arabinose. Thereafter, the resultants were washed once with sterile distilled water and twice with 10% glycerol through a centrifuge to prepare competent cells (Sambrook and Russell 2001). The integrative plasmid, pMu-R6K_pntAB asd aspC-thrO, was electroporated with the prepared competent cells, SOC medium (1 mL) was added thereto, and then the resultants were incubated at 37° C. for 1 hour. Thereafter, the resultants were plated on LB plate medium containing chloramphenicol (25 μg/L), and then colony PCR of chloramphenicol-resistant bacterial colonies was carried out using SEQ ID NOS: 10 and 15 to confirm that the gene, pntAB asd aspC thrO, was introduced into the genome. Thereafter, the plasmid pJW168 was transformed into the selected strain, and then plated on LB plate medium supplemented with 0.1 mM IPTG to remove a chloramphenicol-resistant marker (cat) (Beatri'z Palmeros et al., 2000, Gene). The introduction ofpMu-R6K_gdhA aspC thrO was sequentially carried out by the mini-Mu integration method as described above, and then colony PCR was performed using SEQ ID NOS: 18 and 15 to confirm whether the introduction was successful. In addition, after the introduction ofpMu-ppc aspA thrO, colony PCR was performed using SEQ ID NOS: 22 and 27 to confirm such introduction. The thus-produced wild-type E. coli (W3110)-based threonine-producing strain was named CJT1.

### EXAMPLE 2

### Preparation of ilvA Gene-Deficient Strain

An FRT-one-step-PCR deletion method was used to delete the gene ilvA, which is involved in a representative pathway for threonine degradation (Kirill A, et al., 2000, PNAS). A deletion cassette was prepared by PCR using the primers of SEQ ID NOS: 28 and 29 with the vector pKD3 as a template. Denaturation was performed at 94° C. for 30 seconds, annealing at 55° C. for 30 seconds, and extension at 72° C. for 3 minutes using SolGent™ Pfu-X DNA Polymerase (SolGent, Korea); and this was repeated 30 times.

The resulting PCR product was electrophoresed on a 1.0% agarose gel, and the ilvA-deficient cassette was purified from a band having a size of 1.2 kbp. KCCM10541 pKD46 and CJT1 pKD46, the strains in which the vector pKD46 (Kirill A, et al., 2000, PNAS) was introduced, were cultured on LB medium containing ampicillin (100 μg/L) and 5 mM L-arabinose at 30° C./200 rpm until OD₆₀₀ reached 0.6. Thereafter, the resultants were washed once with sterile distilled water and twice with 10% glycerol to prepare competent cells. The ilvA-deficient cassette was introduced into the prepared competent cells through electroporation (2500 V). Thereafter, SOC medium (1 mL) was added thereto, and then the resultants were cultured overnight at 37° C. The strains showing the resistance were selected.

The selected strains were subjected to colony PCR using the primers of SEQ ID NOS: 28 and 29, and the ilvA gene deficiency was identified by observing that the size of the gene on the 1.0% agarose gel was 1.2 kb. The identified strains were once again transformed into the vector pCP20 (Kirill A, et al., 2000, PNAS), and the final ilvA gene-deficient strain, which had a gene size of 150 bp on the 1.0% agarose gel, was prepared through colony PCR of the colonies, which were obtained by plating the transformed strains on LB plate medium containing ampicillin (100 μg/L), under the same conditions. The strain in which ilvA is deleted from KCCM10541 was named KCCM10541-ilvA; and the strain in which ilvA is deleted from CJT1 was named CJT1-ilvA.

### EXAMPLE 3

### Preparation of cimA Gene-Introduced Strain

DNAs, Pcj1_cimA, Pcj1_cimA 2.0, and Pcj1_cimA 3.7 (Atsumi, Liao J C, Appl Environ Microbiol. 2008), were prepared by codon-optimization based on the gene cimA derived from Methanococcus jannaschii containing the CJ1 promoter (Korean Patent No. 10-0620092). A BamHI site was added to both ends when synthesizing the gene, and then subcloned into the CopyControl pCC1BAC (BamHI) (Epicentre, USA) to prepare the plasmids, pCC1BAC: Pcj1_cimA, pCC1BAC: Pcj1_cimA 2.0, and pCC1BAC: Pcj1_cimA 3.7 which finally express the gene cimA. In addition, the three plasmids above and the control plasmid pCC1BAC were transformed into the strains, KCCM10541-ilvA and CJT1-ilvA, by electroporation (2500 V) to finally prepare strains to be used for the experiments. For the comparison of the experiments, the strains in which pCC1BAC was transformed into the two strains, KCCM10541 and CJT1, in the same manner as described above was also prepared.

In the above, the gene cimA encoded wild-type citramalate synthase; and cimA 2.0 and cimA 3.7 encoded mutant-type citramalate synthase in which specific activities were increased and in which feedback-inhibition by isoleucine was reduced (Atsumi, Liao J C, Appl Environ Microbiol. 2008). The amino acid sequences for cimA 2.0 and cimA 3.7 were SEQ ID NOS: 2 and 3, and the nucleotide sequences corresponding thereto are SEQ ID NOS: 5 and 6, respectively.

Recombinant microorganisms of KCCM10541 pCC1BAC, KCCM10541-ilvA pCC1BAC, KCCM10541-ilvA pCC1BAC:Pcj1_cimA, KCCM10541-ilvA pCC1BAC:Pcj1_cimA 2.0, and KCCM10541-ilvA pCC1BAC:Pcj1_cimA 3.7 were prepared based on KCCM10541 or KCCM10541-ilvA. In addition, recombinant microorganisms of CJT1 pCC1BAC, CJT1-ilvA pCC1BAC, CJT1-ilvA pCC1BAC:Pcj1_cimA, CJT1-ilvA pCC1BAC:Pcj1_cimA 2.0, and CJT1-ilvA pCC1BAC:Pcj1_cimA 3.7 were prepared based on CJT1 or CJT1-ilvA.

The present inventors named the recombinant microorganisms of ‘CJT1-ilvA pCC1BAC:Pcj1_cimA’, ‘CJT1-ilvA pCC1BAC:Pcj1_cimA 2.0’, and ‘CJT1-ilvA pCC1BAC:Pcj1_cimA 3.7’ as ‘CA03-8303’, ‘CA03-8304’, and ‘CA03-8305’, respectively. In addition, these microorganisms were deposited under the Budapest Treaty to the Korean Culture Center of Microorganisms (KCCM) on Dec. 5, 2014, and the microorganisms were designated as ‘KCCM11632P’, ‘KCCM11633P’, and ‘KCCM11634P’, respectively.

### EXAMPLE 4

### Confirmation of Threonine Productivity by Flask Evaluation of i/vA-Deficient and cimA-Expressing Strains

Flask evaluation was performed in order to compare the amount of threonine produced in the strains prepared in Example 3 above. In the flask test, each of the strains was streaked on a LB plate containing chloramphenicol (25 μg/L) and cultured in an incubator (33° C.) for 16 hours. Thereafter, a single colony was inoculated in LB medium (2 mL), and the resultants were cultured in an incubator (200 rpm/33° C.) for 12 hours. In addition, the threonine-producing flask medium (25 mL) shown in Table 3 below was added to a flask (250 mL), and 500 μL of the medium solution previously cultured was added thereto. Thereafter, the flask was incubated in an incubator (200 rpm/33° C.) for 48 hours, and then the amount of threonine obtained from each strain was compared. The results are shown in Table 4 below.

TABLE 3
Composition of threonine-producing flask medium

| | Concentration | |
|---|---|---|
| Composite | (per Liter) | |
| Glucose | 70 | g |
| Ammonium Sulfate | 25 | g |
| KH₂PO₄ | 1 | g |
| MgSO₄ · 7H₂O | 0.5 | g |
| FeSO₄ · 7H₂O | 5 | mg |
| MnSO₄ · 8H₂O | 5 | mg |
| ZnSO₄ | 5 | mg |
| Calcium carbonate | 30 | g |
| Yeast extract | 2 | g |
| Methionine | 0.3 | g |

| | |
|---|---|
| pH | 6.8 |

TABLE 4
Production of threonine in the producing-strain by flask culture

| | | Amount of | Amount of | Amount of |
|---|---|---|---|---|
| | | sugar | threonine | Acetate |
| | | consumed | produced | produced |
| Strain | OD | (g/L) | (g/L) | (g/L) |
| KCCM10541 | 21.6 | 70 | 34.7 | 0.35 |
| pCC1BAC | | | | |
| KCCM10541-ilvA | 5.5 | 12.0 | 6.6 | 0.11 |
| pCC1BAC | | | | |
| KCCM10541-ilvA | 18.5 | 70 | 37.5 | 0.15 |
| pCC1BAC:Pcj1_cimA | | | | |
| KCCM10541-ilvA | 22.5 | 70 | 38.3 | 0.10 |
| pCC1BAC:Pcj1_cimA | | | | |
| 2.0 | | | | |
| KCCM10541-ilvA | 23.2 | 70 | 37.8 | 0.08 |
| pCC1BAC:Pcj1_cimA | | | | |
| 3.7 | | | | |
| CJT1 | 35.2 | 70 | 11.4 | 2.15 |
| pCC1BAC | | | | |
| CJT1-ilvA | 6.4 | 13.6 | 3.0 | 1.24 |
| pCC1BAC | | | | |
| CJT1-ilvA | 30.6 | 70 | 14.3 | 1.42 |
| pCC1BAC:Pcj1_cimA | | | | |
| CJT1-ilvA | 36.0 | 70 | 14.7 | 1.32 |
| pCC1BAC:Pcj1_cimA | | | | |
| 2.0 | | | | |
| CJT1-ilvA | 37.0 | 70 | 14.5 | 1.34 |
| pCC1BAC:Pcj1_cimA | | | | |
| 3.7 | | | | |

As shown in Table 4 above, it was observed that the two strains KCCM10541-ilvA pCC1BAC and CJT1-ilvA pCC1BAC, in which ilvA was deleted, did not grow well due to the requirement of isoleucine compared to strains KCCM10541 pCC1BAC and CJT1 pCC1BAC, in which ilvA was not deleted (see OD values). However, it was confirmed that in KCCM10541-ilvA pCC1BAC:Pcj1_cimA, KCCM10541-ilvA pCC1BAC:Pcj1_cimA 2.0, KCCM10541-ilvA pCC1BAC:Pcj1_cimA 3.7, CJT1-ilvA pCC1BAC:Pcj1_cimA, CJT1-ilvA pCC1BAC:Pcj1_cimA 2.0, and CJT1-ilvA pCC1BAC:Pcj1_cimA 3.7, which are the variant strains in which three types of cimA were introduced, the growth was completely recovered and the amount of threonine produced was increased by about 10% to 30%, while the amount of acetate produced was reduced by about 30% to 60%.

In particular, it was confirmed that when cimA 2.0 or cimA 3.7 was introduced, the strain growth ability was increased by 20% or higher and the amount of threonine produced was slightly increased, while the amount of acetate produced was further reduced, compared to the case in which cimA was introduced into the i/vA-deficient strains.

From the foregoing, one of ordinary skill in the art to which the present disclosure pertains will be able to understand that the present disclosure may be embodied in other specific forms without modifying the technical concepts or essential characteristics of the present disclosure. In this regard, the exemplary embodiments disclosed herein are only for illustrative purposes and should not be construed as limiting the scope of the present disclosure. On the contrary, the present disclosure is intended to cover not only the exemplary embodiments but also various alternatives, modifications, equivalents, and other embodiments that may be included within the spirit and scope of the present disclosure as defined by the appended claims.

1. A recombinant microorganism producing threonine, wherein an activity of threonine deaminase (IlvA) is reduced or inactivated and an activity of citramalate synthase is retained.

2. The recombinant microorganism of claim 1, wherein the threonine deaminase is represented by an amino acid sequence of SEQ ID NO: 7.

3. The recombinant microorganism of claim 1, wherein the citramalate synthase is citramalate synthase derived from Methanococcus jannaschii.

4. The recombinant microorganism of claim 1, wherein the citramalate synthase is represented by an amino acid sequence selected from the group consisting of SEQ ID NOS: 1, 2, and 3.

5. The recombinant microorganism of claim 1, wherein the recombinant microorganism is a microorganism of the genus Corynebacterium or a microorganism of the genus Escherichia.

6. The recombinant microorganism of claim 5, wherein the microorganism of the genus Corynebacterium is Corynebacterium glutamicum and the microorganism of the genus Escherichia is Escherichia coli.

7. A method for producing threonine, comprising:
culturing the recombinant microorganism of claim 1 in a medium; and
recovering threonine from the microorganism or the cultured medium.
